(19) United States
(12) Patent Application Publication
(10) Pub. No.: US 2024/0206823 A1
(43) Pub. Date: Jun. 27, 2024

(54) METHOD, APPARATUS AND PROGRAM FOR CALCULATING DISEASE RISK BASED ON HEART RATE MEASUREMENT

(71) Applicant: TOBEDTX, Seoul (KR)

(72) Inventors: Yang Koo KANG, Seoul (KR); Ki Doo NAM, Incheon (KR)

(73) Assignee: TOBEDTX, Seoul (KR)

(21) Appl. No.: 18/312,983

(22) Filed: May 5, 2023

(30) Foreign Application Priority Data
Dec. 23, 2022 (KR) 10-2022-0183649

(51) Int. Cl. A61B 5/00; A61B 5/024

(52) U.S. Cl. CPC A61B 5/7275; A61B 5/02405; A61B 5/7267

(57) ABSTRACT

Disclosed is a method of calculating a disease risk based on a heart rate measurement, comprising the steps of: acquiring heart rate information measured from one or more heart rate sensors; extracting one or more parameters from the heart rate information; analyzing the extracted parameters using a pre-trained model; and calculating a risk for one or more diseases based on the analysis result.

### CROSS-REFERENCE TO RELATED APPLICATION

This application claims priority to and the benefit of Korean Patent Application No. 10-2022-0183649, filed on Dec. 23, 2022, the disclosure of which is incorporated herein by reference in its entirety.

### BACKGROUND

### 1. Field of the Invention

The present invention relates to a method, a device, and a program for calculating a disease risk based on a heart rate measurement, and more specifically, to a method of determining one or more diseases of a user by using measured heart rate information of the user.

### 2. Discussion of Related Art

Previously, in order to determine various autonomic nervous system diseases held by users, it was necessary to go through a complicated procedure using expensive equipment held by hospitals, and there was a problem that it was difficult for users to grasp information on their own diseases in a timely manner.

Accordingly, several technologies for determining a disease from the user's biometric information using the artificial intelligence technology have been developed, but there are problems that are limited to a specific disease, low accuracy, and a complicated process of acquiring the user's biometric information.

Accordingly, there is a need to develop a technology capable of conveniently and accurately determining various diseases of a user.

### SUMMARY OF THE INVENTION

The problem to be solved by the present disclosure is to provide a method, device, and program for calculating a disease risk based on heart rate measurement.

The problems to be solved by the present disclosure are not limited to the above-mentioned problems, and other problems not mentioned will be clearly understood by the skilled person in the art from the following description.

According to an embodiment of the present invention, a method of calculating a disease risk based on a heart rate measurement comprises the steps of: obtaining heart rate information measured from one or more heart rate sensors; extracting one or more parameters from the heart rate information; analyzing the extracted parameters using a pre-trained model; and calculating a risk of one or more diseases based on the analysis result.

To achieve these objects and other advantages and in accordance with the purpose of the invention, as embodied and broadly described herein, a device for calculating a disease risk based on a heart rate measurement includes a memory configured to store one or more instructions and a processor configured to execute the one or more instructions stored in the memory, wherein the processor executes the one or more instructions to perform a method of calculating a disease risk based on a heart rate measurement.

In order to solve the above-described problems, a disease risk calculation program based on heart rate measurement according to an embodiment of the present disclosure may be combined with a computer that is hardware, and may be stored in a computer-readable recording medium so as to perform a method of calculating a disease risk based on heart rate measurement.

Other detailed matters of the present invention are included in the detailed description and the drawings.

### BRIEF DESCRIPTION OF THE DRAWINGS

FIG. 1 is a diagram illustrating a system for calculating a disease risk based on heart rate measurement according to an embodiment of the present invention.

FIG. 2 is a hardware block diagram of the device for calculating a disease risk based on heart rate measurement according to an embodiment of the present invention.

FIG. 3 is a diagram illustrating a method of calculating a disease risk based on heart rate measurement according to an embodiment of the present invention.

FIG. 4 is a diagram illustrating a method of calculating a disease risk based on heart rate measurement according to another embodiment of the present invention.

### DETAILED DESCRIPTION OF EXEMPLARY EMBODIMENTS

Advantages and features of the present invention and methods of achieving the same will become apparent with reference to embodiments described in detail below together with the accompanying drawings. However, the present invention is not limited to the embodiments disclosed below, but may be implemented in various different forms, and the present embodiments are provided so that the disclosure of the present invention is complete and the scope of the present invention is completely known to a skilled person in the art in the technical field to which the present invention belongs, and the present invention is defined only by the scope of the claims.

The terminology used herein is for the purpose of describing embodiments and is not intended to be limiting of the present invention. In the specification, a singular form includes a plural form unless specifically mentioned in the text. The terms “comprises” and/or “comprising” used in the specification do not exclude the presence or addition of one or more other constituent elements in addition to the mentioned constituent elements. Throughout the specification, like reference numerals refer to like feature elements, and “and/or” includes each and every combination of the stated elements. Although “first”, “second”, and the like are used to describe various components, these components are not limited by these terms. These terms are only used to distinguish one component from another. Therefore, it goes without saying that the first component mentioned below may be the second component within the technical spirit of the present invention.

Unless otherwise defined, all terms (including technical and scientific terms) used herein have the same meaning as commonly understood by skilled people in the art in the technical field to which the present invention belongs. In addition, terms defined in commonly used dictionaries are not ideally or excessively interpreted unless they are clearly specifically defined.

As used herein, the term “unit” or “module” refers to software, a hardware component such as FPGA or ASIC, and the term “unit” or “module” performs certain roles. However, the term “unit” or “module” is not limited to software or hardware. The “unit” or “module” may be configured to be in an addressable storage medium, or may be configured to reproduce one or more processors. Thus, for example, the “unit” or “module” includes components such as software components, object-oriented software components, class components, and task components, processes, functions, attributes, procedures, subroutines, segments of program code, drivers, firmware, microcode, circuits, data, databases, data structures, tables, arrays, and variables. The functionality provided within the components and “units” or “modules” may be combined into a smaller number of components and “units” or “modules” or further separated into additional components and “units” or “modules”.

In the present specification, a computer means all kinds of hardware devices including at least one processor, and may be understood as encompassing a software configuration operating in a corresponding hardware device according to an embodiment. For example, a computer may be understood as including all of a smartphone, a tablet PC, a desktop, a notebook computer, and a user client and an application driven in each device, but is not limited thereto.

Hereinafter, embodiments of the present disclosure will be described in detail with reference to the accompanying drawings.

Although each step described in the specification is described as being performed by a computer, the subject of each step is not limited thereto, and according to an embodiment, at least a part of each step may be performed in different devices.

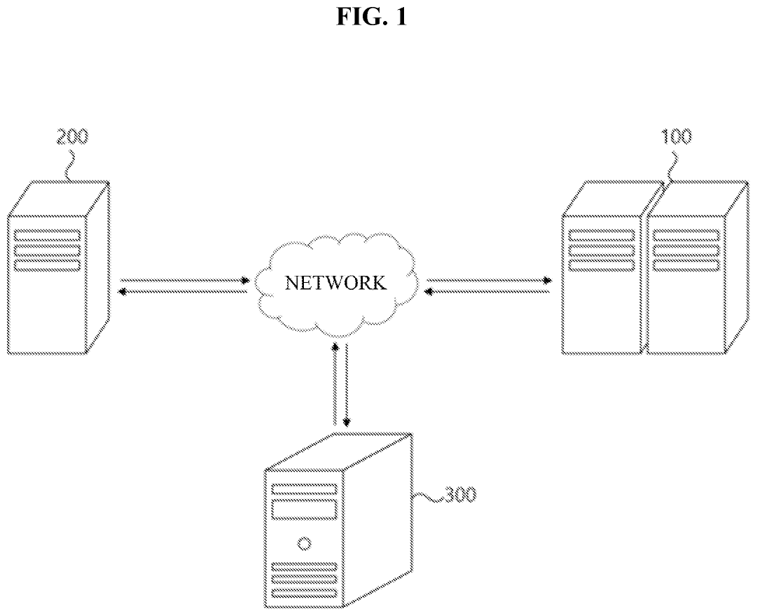

FIG. 1 is a diagram illustrating a system for calculating a disease risk based on heart rate measurement according to an embodiment of the present invention.

Referring to FIG. 1, the system for calculating a disease risk based on heart rate measurement according to an embodiment of the present disclosure may include a determination device 100, a user terminal 200, and an external server 300.

Here, the system for calculating a disease risk based on heart rate measurement illustrated in FIG. 1 is according to an embodiment, and the feature element thereof is not limited to the embodiment illustrated in FIG. 1, and may be added, changed, or deleted as necessary.

The determination device 100 may obtain heart rate information measured from one or more heart rate sensors, extract one or more parameters from the heart rate information, analyze the extracted parameters using a pre-trained model, and calculate a risk level for one or more diseases based on a result of the analysis.

The user terminal 200 may access a website through a network, and may be provided with a disease risk calculation service based on a heart rate measurement from the determination device 100.

The user terminal 200 may include at least one of a smartphone including a display, a tablet PC, a desktop computer, and a notebook computer in at least a part of the user terminal 200, and may be provided with a disease risk calculation service based on heart rate measurement provided from the determination device 100 in a process of executing a browser. However, it is not limited thereto.

The external server 300 may be connected to the determination device 100 through a network, and the determination device 100 may store and manage various types of information for performing a method of calculating a disease risk based on a heart rate measurement.

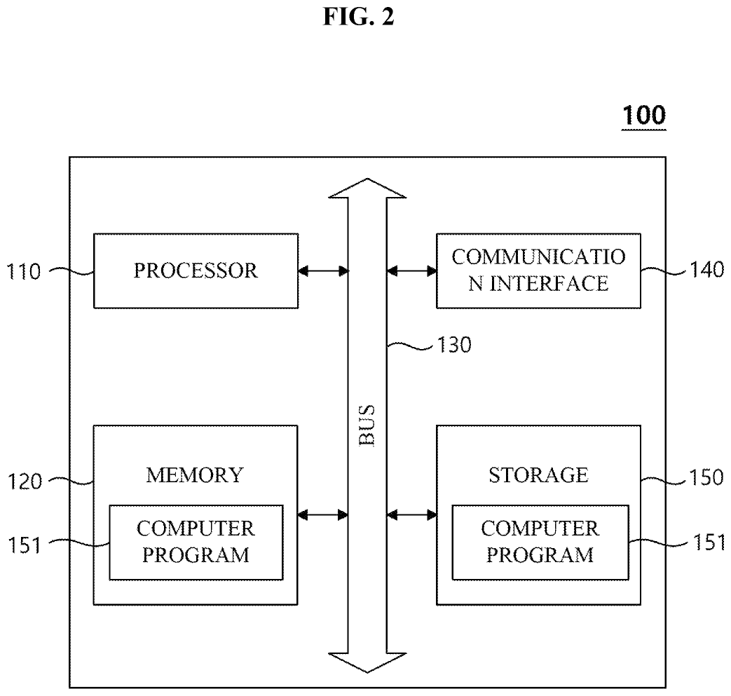

In addition, the external server 300 may receive and store various pieces of information and data generated as the determination device 100 performs the method of calculating a disease risk based on a heart rate measurement. For example, the external server 300 may be a storage server separately provided outside the determination device 100. Referring to FIG. 2, hardware feature of the determination device 100 will be described.

FIG. 2 is a hardware configuration diagram of a determination device according to an embodiment of the present invention.

Referring to FIG. 2, the determination device 100 (hereinafter, referred to as a computing device) according to an embodiment of the present disclosure may include one or more processors 110, a memory 120 for loading a computer program 151 executed by the processors 110, a bus 130, a communication interface 140, and a storage 150 for storing the computer program 151. Here, only components related to the embodiment of the present invention are shown in FIG. 2. Therefore, it may be understood by those skilled in the art that other general-purpose feature elements may be further included in addition to the elements illustrated in FIG. 2.

The processor 110 controls the overall operation of each feature of the computing device 100. The processor 110 may include a central processing unit (CPU), a micro processor unit (MPU), a micro controller unit (MCU), a graphic processing unit (GPU), or any type of processor well known in the technical field of the present invention.

In addition, the processor 110 may perform an operation on at least one application or program for executing a method according to embodiments of the present disclosure, and the computing device 100 may include one or more processors.

In various embodiments, the processor 110 may further include a Random Access Memory (RAM) (not shown) and a Read-Only Memory (ROM, not shown) for temporarily and/or permanently storing signals (or data) processed in the processor 110. In addition, the processor 110 may be implemented in the form of a system on chip (SoC) including at least one of a graphic processor, a RAM, and a ROM.

The memory 120 stores various data, commands, and/or information. The memory 120 may load the computer program 151 from the storage 150 to execute the method/operation according to various embodiments of the present disclosure. When the computer program 151 is loaded into the memory 120, the processor 110 may perform the method/operation by executing one or more instructions constituting the computer program 151. The memory 120 may be implemented as a volatile memory such as a RAM, but the technical scope of the present disclosure is not limited thereto.

The bus 130 provides a communication function between feature elements of the computing device 100. The bus 130 may be implemented as various types of buses such as an address bus, a data bus, and a control bus.

The communication interface 140 supports wired/wireless Internet communication of the computing device 100. Also, the communication interface 140 may support various communication methods other than Internet communication. To this end, the communication interface 140 may include a communication module well known in the technical field of the present invention. In some embodiments, the communication interface 140 may be omitted.

The storage 150 may non-temporarily store the computer program 151. When the method of calculating the disease risk using the heart rate information is performed through the computing device 100, the storage 150 may store various information necessary for providing the method of calculating the disease risk using the heart rate information.

The storage 150 may be configured to include a non-volatile memory such as a read only memory (ROM), an erasable programmable ROM (EPROM), an electrically erasable programmable ROM (EEPROM), a non-volatile memory such as flash memory, a hard disk, a removable disk, or any form of computer-readable recording medium well known in the technical field to which the present invention pertains.

The computer program 151 may include one or more instructions that, when loaded into the memory 120, cause the processor 110 to perform a method/operation according to various embodiments of the present disclosure. That is, the processor 110 may perform the method/operation according to various embodiments of the disclosure by executing the one or more instructions.

In an embodiment, the computer program 151 may include one or more instructions for performing a method of calculating a disease risk based on heart rate measurement, the method including: obtaining heart rate information measured by one or more heart rate sensors; extracting one or more parameters from the heart rate information; analyzing the extracted parameters using a pre-trained model; and calculating a risk for one or more diseases based on a result of the analysis.

The steps of the method or algorithm described in relation to the embodiment of the present disclosure may be implemented directly in hardware, implemented in a software module executed by hardware, or implemented in a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any form of computer-readable recording medium well known in the art to which the present invention pertains.

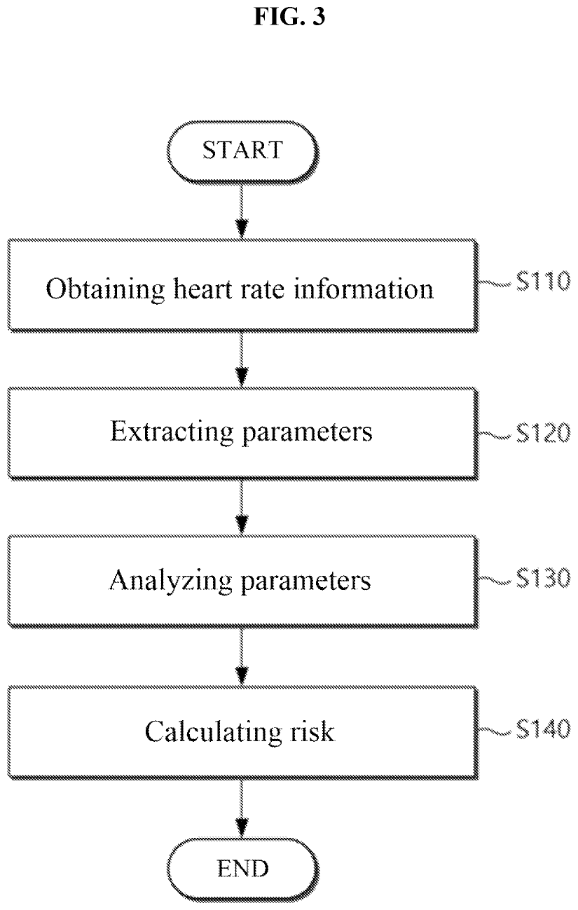

The feature elements of the present invention may be implemented as a program (or an application) to be executed in combination with a computer that is hardware, and may be stored in a medium. The feature elements of the present invention may be implemented using software programming or software elements, and similarly, the embodiments may be implemented with any programming or scripting language such as C, C++, Java, assembler language, or the like, with the various algorithms being implemented with any combination of data structures, objects, processes, routines or other programming elements. Functional aspects may be implemented with algorithms executed on one or more processors. Hereinafter, referring to FIG. 3, a method of calculating a disease risk level based on a heart rate measurement provided by the computing device 100 will be described.

FIG. 3 is a diagram illustrating a method of calculating a disease risk based on heart rate measurement according to an embodiment of the present invention.

Referring to FIG. 3, the computing device 100 may obtain heart rate information measured from one or more heart rate sensors (S110).

In various embodiments, the computing device 100 may collect data obtained by sensing the heart rate information of the user from a sensor attached to the body of the user or a device that measures the heart rate information of the tester in a non-contact manner at a position spaced apart from the user. For example, the user may wear a wearable device on a hand, a head, or the like, or may attach a sensor capable of measuring separate heart rate information to the body of the user, and the sensor that contacts the body of the user may periodically measure the heart rate information of the user and transmit the measured heart rate information to the computing device 100.

In various embodiments, a device including a sensor may be manufactured in a kiosk form and may collect user's heart rate information. For example, a kiosk may be provided in a public facility, and the user may measure heart rate information through the kiosk and receive a disease risk analysis result.

As another example, the method may further include analyzing heart rate information of the user from an image obtained by photographing the user by a camera installed at a position spaced apart from the user, analyzing heart rate information of the user from a voice of the user obtained by a microphone installed at a position spaced apart from the user, and analyzing the heart rate information of the user from an ultrasonic wave returning after being emitted to the user from an ultrasonic device installed at a position spaced apart from the user.

Analyzing the heart rate information through the camera, the microphone, and the ultrasonic device may be performed using at least one device. Meanwhile, each of the camera, the microphone, and the ultrasound device may analyze the user's heart rate information by using the acquired data, transmit the acquired data to a separate external server, and analyze the user's heart rate information by using the data acquired from the external server. Here, the heart rate information may include a heart rate, a heart rate time, a heart rate interval, and the like, but is not limited thereto.

In various embodiments, the computing device 100 may additionally obtain basic information of the user for analysis.

The basic information of the user may include physical information including gender, age, height, weight, and blood pressure, and disease information including disease information of the user. The computing device 100 may provide an instruction for inputting basic information to the user terminal 200 of the user, and may obtain the basic information of the user input through the user terminal 200.

Meanwhile, the computing device 100 may provide an instruction for inputting basic information to the user terminal 200 of an administrator who manages a process of calculating a disease risk of the user, and acquire the basic information of the user that the administrator inputs through the user terminal 200. Meanwhile, the manager may be an expert such as a doctor who may determine a disease risk of the user, but is not limited thereto.

The computing device 100 may extract one or more parameters from the heart rate information (S120).

For example, the one or more parameters may include at least one of a heart rate, a heart rate variability, an electrocardiogram, and one or more parameters derived from the electrocardiogram, but are not limited thereto.

In various embodiments, the computing device 100 may extract, from the heart rate information, an average, a maximum, a minimum, a standard deviation of all NN intervals (SDNN), a standard deviation of differences between adjacent NN intervals (SDSD), the square root of the mean of the sum of the squares of differences between adjacent NN intervals (RMSSD), a count divided by the total number of all NN intervals (PNN50), a count Number of pairs of adjacent NN intervals differing by more than 50 ms in the entire recording (NN50), a count divided by the total number of all NN intervals (PNN20), a count Number of pairs of adjacent NN intervals differing by more than 20 ms in the entire recording (NN20), or a percentile value according to a specific criterion.

In addition, the computing device 100 may extract total power, very low frequency bands power (VLF), low frequency bands power (LF), high frequency bands power (HF), LF/HF, normalized lf power (NLF), normalized hf power (NHF) values, and the like in the frequency domain from the heart rate information, and may also extract various values including C. HRV triangular index, sampen, Cardiac Pathmatic Index (CSI), Cadiac Vagal Index (CVI), and Modified CSI, but is not limited thereto.

The computing device 100 may analyze the parameter extracted in operation S120 by using a pre-trained model. (S130)

Also, the computing device 100 may calculate a risk for one or more diseases based on the analysis result of operation S130. (S140)

In various embodiments, the pre-trained model may be an artificial intelligence model including one or more network functions, but is not limited thereto.

In an embodiment, the computing device 100 may generate input data by using the extracted parameter. The input data may include one or more feature values derived from the parameter. A specific method of deriving the feature value from the parameter is not limited and may be performed according to a commonly known technique.

In an embodiment, the computing device 100 may input the generated input data to a pre-trained artificial intelligence model. The computing device 100 may obtain an output of the artificial intelligence model and determine a risk for one or more diseases for the user based on the output.

In various embodiments, the one or more diseases may refer to an autonomic nervous system disease of the user. For example, the autonomic nervous system disease may include at least one of arrhythmia, epilepsy, stroke, acute myocardial infarction, and acute pneumonia, but is not limited thereto.

In various embodiments, the artificial intelligence model may output a value indicating a risk for one or more diseases. For example, the artificial intelligence model may output a value of the same number as the number of diseases to be determined, and each value may indicate a risk level of the disease to be determined. For example, the risk may be output as a value of 0 to 1, but is not limited thereto.

In order to train such an artificial intelligence model, the computing device 100 may obtain medical information about various patients. Each medical information may include heart rate information of a patient and information about a disease possessed by the corresponding patient or details treated for each disease.

The computing device 100 may extract input data for learning from heart rate information of each patient, and output data for learning corresponding to each input data may include information on a disease possessed by the corresponding patient. For example, when an artificial intelligence model capable of determining arrhythmia, epilepsy, and myocardial infarction is trained, output data may be generated as (1, 0, 1) when the corresponding patient has a history of arrhythmia and myocardial infarction, but is not limited thereto.

In various embodiments, when the corresponding patient has not been diagnosed with epilepsy but has a record of being treated for epilepsy diagnosis, the risk may be additionally calculated based on the number of treatments, treatment results, treatment period, and the like. For example, if the subject is treated under the finding that epilepsy is suspected but epilepsy is not diagnosed, the output data may be generated as (1, 0.5, 1), but is not limited thereto.

The computing device 100 may train the artificial intelligence model based on the input data and the output data for learning.

In various embodiments, the artificial intelligence model may calculate a plurality of disease risks by one model, but a plurality of models may be used. For example, a plurality of models for calculating the risk of each disease using the same input data may be used.

The computing device 100 may analyze a disease risk of a corresponding user by synthesizing result values output from a plurality of models.

In various embodiments, the computing device 100 may ensemble result values output by a plurality of models. For example, an average value may be derived by analyzing result values of a plurality of models that calculate the risk of the same disease, or a disease risk of the patient may be determined using the result values calculated by the plurality of models.

In various configurations, a plurality of models capable of calculating a plurality of disease risks may be used. For example, the risk of each disease may be calculated by using a first model for predicting the risk of arrhythmia and epilepsy, a second model for predicting the risk of epilepsy and myocardial infarction, and a third model for predicting the risk of myocardial infarction and arrhythmia, and the risk of the disease of the patient may be determined by synthesizing the results for each disease. For example, the risk may be determined by averaging the risk degrees derived from different models for a specific disease, or as another example, it may be determined that both models predict the risk (e.g., the calculated risk degree is equal to or greater than a predetermined reference value) to determine that the risk is dangerous, when only one model predicts the risk, it may be determined that the possibility of suspicion is present, and when both models are determined not to be dangerous, it may be determined that the risk is low or absent. Through this, it is possible to obtain a determination result that comprehensively reflects the correlation between different diseases.

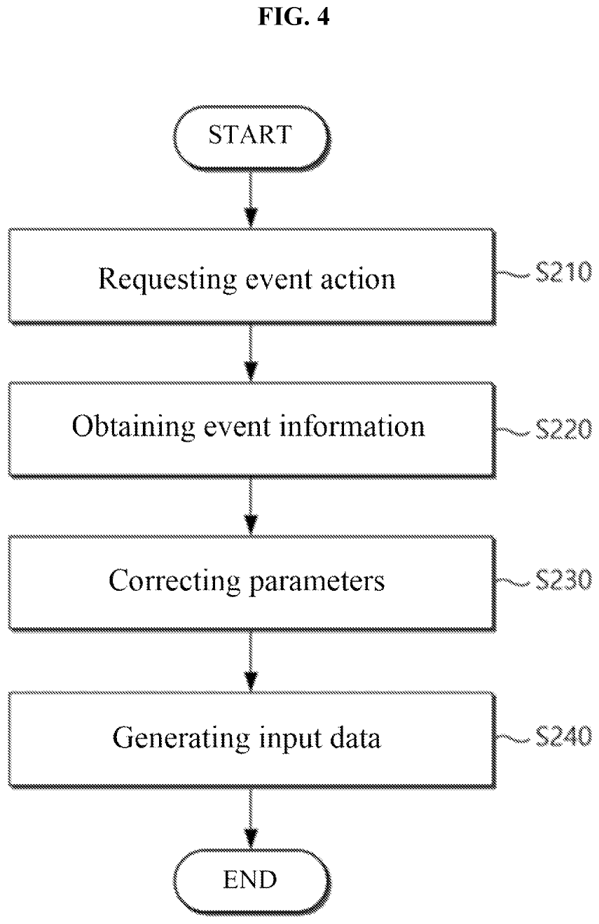

FIG. 4 is a diagram illustrating a method of calculating a disease risk based on heart rate measurement according to another embodiment of the present invention.

In operation S210, the computing device 100 may determine an event for obtaining heart rate information, and may request the user to perform an action corresponding to the event.

The computing device 100 may acquire information on the disease to be analyzed and acquire behavior information of the user required to analyze the disease. Thereafter, the computing device 100 may output information that requests the user to perform the corresponding action.

For example, the computing device 100 may request the user to perform various actions such as deep breathing, Valsalva maneuver breathing, or sitting and standing up.

In operation S220, the computing device 100 may obtain information about an event that occurs while measuring the user's heart rate information.

In various embodiments, the computing device 100 may provide an instruction for inputting activity information on an activity performed by the user to the user terminal 200, and may obtain the activity information through the user terminal 200. The activity information may include sleep, meals, walking, standing up, and the like, and may include a breathing state such as comfortable breathing, fast breathing, and Valsalva maneuver breathing. Also, the computing device 100 may obtain activity information of the user by using a sensor of the wearable device.

In addition, the computing device 100 may obtain the activity information of the user from a camera that photographs the user or a sensor that senses the motion of the user, and may obtain the activity information of the user at the time when the heart rate information is measured by using data that photographs or senses the motion of the user corresponding to the time when the heart rate information of the user is measured.

In various embodiments, the disease risk calculating method according to the present disclosure may be frequently performed by using information collected while the user performs daily life.

For example, after allowing the user to perform a natural daily life in an environment (e.g., an environment in which the user wears a wearable device or in which a non-contact heart rate information measuring device is installed) in which heart rate information may be measured in a contact or non-contact manner, information about what activity the user has performed during a corresponding period may be obtained through a questionnaire at a specific time point. Alternatively, the behavior information of the user corresponding to the heart rate information may be obtained using a wearable device worn by the user.

The computing device 100 may acquire the collected heart rate information and information on the user's behavior corresponding to each time point. Through this, the computing device 100 may acquire heart rate information according to the behavior acquired while the user performs daily life, process the heart rate information, and acquire input data that can be input to the artificial intelligence model or acquire basic data for statistical analysis.

The computing device 100 may input the obtained data to the artificial intelligence model to obtain an output, or may calculate various disease risks of the user through a predetermined statistical analysis method or comparison with an existing database.

In addition, the computing device 100 may guide an activity to be performed by the user, collect heart rate information of the user while the user performs the corresponding activity, and acquire activity information performed by the user in a process of collecting the heart rate information.

In various embodiments, the computing device 100 may determine various disease risks of the user by analyzing basic information, heart rate information, and activity information of the user.

In various embodiments, the computing device 100 may correct at least a part of the parameter extracted from the heart rate information of the user by using event information corresponding to the behavior performed by the user (S230). For example, since a basic heart rate when the user stands still may be different from a basic heart rate when the user moves, the computing device 100 may perform an amendment in consideration of this.

In various embodiments, the computing device 100 may generate a parameter value according to event information corresponding to an activity performed by the user, and may generate input data including the corresponding parameter value (S240).

In detail, the computing device 100 may extract input data from the basic information, the heart rate information, and the activity information, and may input the input data to the pre-trained artificial intelligence model. The computing device 100 may convert the basic information, the heart rate information, and the activity information into the form of input data in order to input the same into a pre-trained artificial intelligence model, and may input the converted input data into the artificial intelligence model.

The computing device 100 may obtain an output of the artificial intelligence model and determine various disease risks of the user based on the output. The AI model may output an output value for input data according to a learned result, and the computing device 100 may determine various disease risks of the user based on the output value.

The computing device 100 may extract input data of the artificial intelligence model from the basic information, the heart rate information, and the activity information. The input data may be configured in the form of a vector including values extracted based on at least some of the basic information, heart rate information, and activity information, but is not limited thereto.

In addition, the user's basic information and activity information may be converted into a specific value according to a predetermined criterion.

The computing device 100 may train the artificial intelligence model by using input data including the extracted values and learning data in which whether various diseases corresponding to the input data are present is labeled.

The trained artificial intelligence model may output various disease risks corresponding to input data extracted from user information.

In various embodiments, the computing device 100 may generate a plurality of input data for each operation of the user. For example, the user may stand, lie down, sit, stand up, and breathe in Valsalva maneuver during the measurement period. In this case, the computing device 100 may generate input data according to the heart rate information for each operation time point of the user.

In addition, the computing device 100 may define an additional operation corresponding to the change in the operation when the operation of the user is changed. For example, when the user stands up while sitting, an additional operation of “sitting and then waking up” may be defined, and another input data based on heart rate information for a predetermined time after the corresponding operation change may be generated.

The computing device 100 may obtain output data obtained through the artificial intelligence model from each input data. The computing device 100 may assign a weight according to an operation of input data corresponding to each output data to each output data. For example, a high weight may be given to an operation in which the arrhythmia is well detected. The computing device 100 may determine whether the user has arrhythmia, based on weighted output data (e.g., deriving an average value, counting the number of output data exceeding a predetermined reference value, comparing the counted number with a reference value for determining arrhythmia, etc.).

In various embodiments, the computing device 100 may calculate a first arrhythmia score of the user based on output data obtained from input data corresponding to the stationary operation, and may calculate a second arrhythmia score of the user based on output data obtained from input data corresponding to the operation change time point.

The computing device 100 may determine that the user is in an arrhythmia when both the first arrhythmia score and the second arrhythmia score are equal to or greater than a predetermined reference value, determine that the user is in an arrhythmia risk group when the second arrhythmia score is equal to or greater than the predetermined reference value but the first arrhythmia score is less than the predetermined reference value, and determine that the user is normal when both scores are less than the predetermined reference value. In addition, when the first arrhythmia score is higher than the second arrhythmia score, re-measurement may be requested.

The present invention may calculate a risk degree for various diseases held by the user by using the user's heart rate information.

Thus, a user can easily and accurately identify the degree of risk of the disease or illness that the user has, and thus can detect or prevent diseases at an early stage.

The effects of the present invention are not limited to the effects mentioned above, and other effects not mentioned may be clearly understood by the skilled person in the art from the following description.

Although the embodiments of the present invention have been described above with reference to the accompanying drawings, it will be understood that the skilled person in the art in the technical field to which the present invention pertains may be implemented in other specific forms without changing the technical idea or essential feature. Therefore, it should be understood that the above-described embodiments are exemplary and not restrictive in all aspects.

What is claimed is:

1. A method of calculating a disease risk based on a heart rate measurement, the method comprising:
obtaining heart rate information measured by at least one heart rate sensor;
extracting at least one parameter from the heart rate information;
analyzing the extracted parameter by using a pre-trained model; and
calculating a risk for at least one disease based on a result of the analyzing.

2. The method of calculating a disease risk based on a heart rate measurement of claim 1, wherein the calculating of the risk for each disease comprises:
generating input data using the extracted parameter;
inputting the input data to a pre-trained artificial intelligence model; and
obtaining an output of the artificial intelligence model and determining the risk for one or more diseases for the user based on the output.

3. The method of calculating a disease risk based on a heart rate measurement of claim 2, wherein the acquiring of the heart rate information comprises acquiring information on an event generated while the heart rate information is measured.

4. The method of calculating a disease risk based on a heart rate measurement of claim 3, wherein the acquiring of the heart rate information comprises:
determining an event for acquiring the heart rate information; and
requesting the user to perform an action corresponding to the event.

5. The method of calculating a disease risk based on a heart rate measurement of claim 3, wherein the generating of the input data comprises generating the input data including a parameter related to the event.

6. The method of calculating a disease risk based on a heart rate measurement of claim 3, wherein the extracting of the parameter comprises correcting at least a part of the parameter based on the information about the event.

7. The method of calculating a disease risk based on a heart rate measurement of claim 1, wherein the calculating of the risk of the at least one disease comprises calculating the risk of the at least one autonomic nervous system-related disease of the user as a feature.

8. The method of calculating a disease risk based on a heart rate measurement of claim 7, wherein the autonomic nervous system disease includes at least one of arrhythmia, epilepsy, stroke, acute myocardial infarction, and acute pneumonia.

9. The method of calculating a disease risk based on a heart rate measurement of claim 1, wherein the at least one parameter comprises at least one of a heart rate, a heart rate variability, an electrocardiogram, and at least one parameter derived from the electrocardiogram.

10. A device comprising:
a memory configured to store one or more instructions; and
a processor configured to execute the one or more instructions stored in the memory, wherein the processor is configured to execute the method of claim 1 by executing the one or more instructions.

11. A non-transitory computer readable storage medium storing instructions/program which, when executed by a hardware computer, cause the computer to carry out the method of claim 1.
